(19) United States
(12) Patent Application Publication
(10) Pub. No.: US 2025/0135180 A1
(43) Pub. Date: May 1, 2025

(54) MULTI-CHANNEL AUTOMATIC INFUSION VALVE

(71) Applicant: Alcon Inc., Fribourg (CH)

(72) Inventors: Parthasarathy PARISHRAM, Irvine, CA (US); Satish YALAMANCHILI, Irvine, CA (US)

(21) Appl. No.: 18/884,665

(22) Filed: Sep. 13, 2024

Related U.S. Application Data
(60) Provisional application No. 63/594,866, filed on Oct. 31, 2023.

(51) Int. Cl. A61M 39/22; A61F 9/007; A61M 3/00; A61M 3/02

(52) U.S. Cl. CPC A61M 39/223; A61F 9/00736; A61M 3/005; A61M 3/0202; A61M 3/022; A61B 2217/007; A61M 2205/3334; A61M 2205/7536

(57) ABSTRACT

The present disclosure generally relates to fluid control valves for delivering and/or aspirating fluid during ophthalmic surgeries and procedures. In one embodiment, a valve assembly includes a first portion configured to fluidly couple with a gas supply line and a second portion configured to fluidly couple with a liquid supply line and an infusion line via two or more through channels. The first portion and the second portion are partitioned or separated from each other by a filter having a hydrophobic membrane configured to prevent the flow of liquids therethrough while allowing the free flow of gas. Accordingly, an infusion liquid may flow through the second portion while gases may be simultaneously aspirated through the first portion, without any liquids travelling into the gas supply line. The gas supply line may thus be utilized to vent or purge gases from the infusion line before or during performance of surgical procedures.

### CROSS-REFERENCE TO RELATED APPLICATIONS

This application claim benefit of and priority to U.S. Provisional Patent Application No. 63/594,866, filed Oct. 31, 2023, which is hereby assigned to the assignee hereof and hereby expressly incorporated by reference in its entirety as if fully set forth below and for all applicable purposes.

### BACKGROUND

Microsurgical procedures frequently require precision cutting, removal, and manipulation of various body tissues. For example, certain ophthalmic surgical procedures, such as pars plana vitrectomies, require cutting and removal of portions of the vitreous humor, a transparent gel-like material that fills the posterior segment of the eye. Simultaneously, while removing the vitreous humor, a liquid solution (e.g., balanced salt solution (BSS)) is typically infused into the eye to maintain intraocular pressure and prevent collapse of the eye wall. In cases of retinal breaks or retinal detachment, the liquid solution may then be exchanged for air, through a process known as fluid-air exchange, to help push out subretinal fluid from the intraocular space while maintaining intraocular pressure and temporarily holding the retina in place. During such procedures, the liquid and air are provided by separate supply lines that are conjoined with a singular downstream infusion line via a stopcock.

In some cases, the air pressure in the gas supply line may build up and cause air to escape into the infusion line, forming air bubbles in the infusion liquid which may travel to the eye and negatively affect intraocular pressure during surgery. Existing designs for check valves of infusion stopcocks, however, do not allow venting through the gas supply line without reverse leakage of liquid and thus, there is currently no effective way to remove the air bubbles or prevent their escape into infusion liquids. Additionally, during some procedures, infusion fluids must be back-flowed through the infusion line in order for other surgical fluids, such as a retinal tamponade, to be injected into the intraocular space. In such cases, the amount of infusion fluid that may be back-flowed is limited by the inability to purge infusion gases through the gas supply line without reverse leakage of liquids therein, which may damage the air pump and/or cause additional complications during fluid infusion.

Therefore, what is needed in the art are improved fluid control valves for ophthalmic fluid infusion that enable aspiration and purging of gases.

### SUMMARY

Aspects of the present disclosure generally relate to devices for ophthalmic procedures, and more particularly, to surgical devices for ophthalmic intraocular fluid delivery (e.g., infusion) and aspiration.

Certain embodiments of the present disclosure provide a valve assembly including a first portion configured to fluidly couple with a gas supply line and a second portion configured to fluidly couple with a liquid supply line and an infusion line. The second portion and the liquid supply line and infusion line are fluidly coupled via two or more through channels. The first portion and the second portion are partitioned or separated from each other by a filter having a hydrophobic membrane configured to prevent the flow of liquids therethrough while allowing the free flow of gas. Accordingly, an infusion liquid may be flowed through the second portion while gases may be simultaneously aspirated into the first portion, without any liquids travelling into the gas supply line. The gas supply line may thus be utilized to vent or purge gases from the infusion line before or during performance of surgical procedures.

The following description and the related drawings set forth herein detail certain illustrative features of one or more embodiments, including those described above.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a perspective view of an exemplary surgical console, according to embodiments described herein.

FIG. 2A illustrates a perspective view of an exemplary valve assembly, according to embodiments described herein.

FIG. 2B illustrates a perspective exploded view of the valve assembly of FIG. 2A, according to embodiments described herein.

FIG. 2C illustrates a schematic cross-sectional view of the valve assembly of FIG. 2A, according to embodiments described herein.

FIG. 2D illustrates a perspective top view of the base of the bottom portion of the valve assembly of FIG. 2A, according to embodiments described herein.

FIG. 2E illustrates a perspective bottom view of the cover of the top portion of the valve assembly of FIG. 2A, according to embodiments described herein.

FIG. 3A illustrates a schematic plan view of an exemplary operational mode of the valve assembly of FIGS. 2A-2E, according to embodiments described herein.

FIG. 3B illustrates a schematic plan view of an exemplary operational mode of the valve assembly of FIGS. 2A-2E, according to embodiments described herein.

FIG. 3C illustrates a schematic plan view of an exemplary operational mode of the valve assembly of FIGS. 2A-2E, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

The present disclosure generally relates to fluid control valves for delivering and/or aspirating fluid during ophthalmic surgeries and procedures. For example, the fluid control valves described herein may be used during vitreoretinal procedures, such as pars plana vitrectomies for the treatment of posterior segment diseases. Vitrectomies typically require cutting and removal of portions of the vitreous humor. In order to maintain intraocular pressure and prevent collapse of the eye during such surgical procedures, liquid is infused into the intraocular space and thereafter aspirated. In certain procedures, the liquid is then exchanged with air or other gases during a process known as fluid-air exchange. During such processes, it is typically beneficial to purge or vent any undesired gases in the infusion line and/or the intraocular space to maintain intraocular pressure. The fluid control valves and methods described herein provide improved structures and mechanisms for infusion fluid flow regulation that enable upstream purging and/or venting of gases from infusion lines while also preventing liquids from the infusion lines to leak into the gas supply lines.

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body and is in proximity to, for example, a surgical console.

In certain embodiments, a valve assembly includes a first portion configured to fluidly couple with a gas supply line and a second portion configured to fluidly couple with a liquid supply line and an infusion line. The second portion and the liquid supply line and infusion line are fluidly coupled via two or more through channels. The first portion and the second portion are partitioned or separated from each other by a filter having a hydrophobic membrane configured to prevent the flow of liquids therethrough while allowing the free flow of gas. Accordingly, an infusion liquid may flow through the second portion while gases may be simultaneously aspirated into the first portion, without any liquids travelling into the gas supply line. The gas supply line may thus be utilized to vent or purge gases from the infusion line before or during performance of surgical procedures.

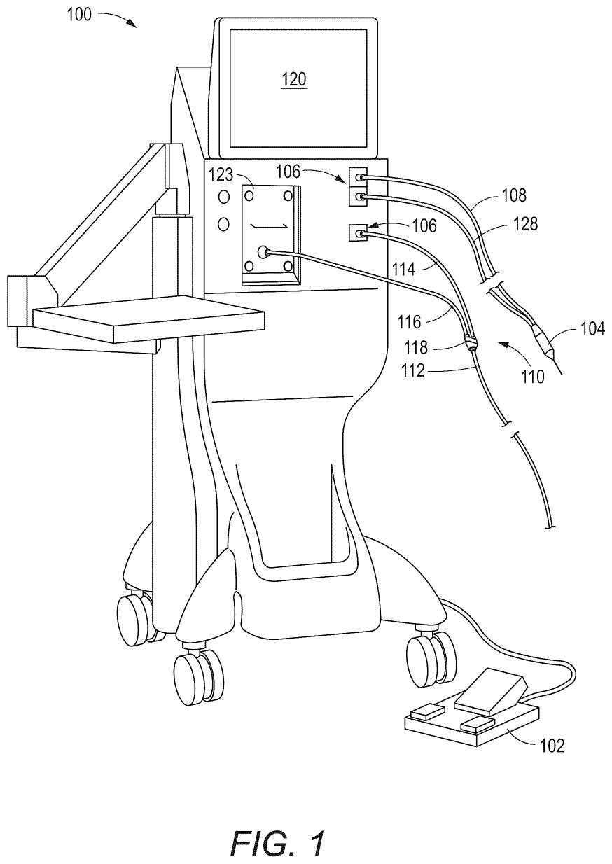

FIG. 1 illustrates a perspective view of an exemplary surgical console 100 that may be utilized in combination with the fluid control valves described herein. The surgical console 100 is operably coupled, physically or wirelessly, to any number of user interfaces, including a foot controller 102 and a surgical tool 104, such as a vitrector. The surgical console 100 provides one or more port connectors 106 for physically coupling the user interfaces to various components or subcomponents of the surgical console 100. For example, the surgical tool 104 may be fluidly coupled with a pneumatic source of the surgical console 100, via a pneumatic line 128 connected to a port connector 106, to facilitate a reciprocating motion of a cutter of the surgical tool 104 for cutting vitreous in a patient's eye. Further, the surgical tool 104 may be fluidly coupled with a vacuum source, via a vacuum supply line 108 connected to a port connector 106, to enable aspiration of cut vitreous from the patient's eye.

Similarly, one or more port connectors 106 may be utilized to couple a fluid infusion system 110 with one or more infusion fluid sources, (e.g., an air/gas source, a liquid perfluorocarbon source, a silicone oil infusion (SOI) source, a BSS source, etc.) to enable infusion of fluids into the eye during vitreous removal. In certain embodiments, fluid supply line 116 of the fluid infusion system 110 may be coupled to one or more infusion fluid sources via a fluidics subsystem 123. As shown in FIG. 1, the fluid infusion system 110 includes an infusion line 112 fluidly coupled with a gas supply line 114 and a separate liquid supply line 116 at a three-way automatic valve assembly 118, which may enable selective flow of different infusion fluids through the infusion line 112.

In operation, the user may control an aspect or mechanism of the surgical tool 104 and/or the fluid infusion system 110 via actuation of the foot controller 102, which may include a foot pedal, or other user input device. For example, the user may press down on (e.g., depress) the foot controller 102 to initiate and increase a flow rate of an infusion fluid from a fluid source through the fluid infusion system 110 and into the eye of the patient. Alternatively, reducing depression of the foot controller 102 (e.g., lifting the user's foot) may decrease and ultimately stop the flow of fluid through the fluid infusion system 110. Accordingly, in certain embodiments, the flow rate of infusion fluids through the fluid infusion system 110 corresponds to the amount of depression of the foot controller 102. In certain embodiments, the surgical console 100 further includes a display 120 for displaying information to the user (the display may also incorporate a touchscreen for receiving user input). Thus, the display 120 may display information about infusion fluid parameters, such as infusion fluid flow rates and intraocular pressure, to the user during operation thereof, as well as information related to the performance of the surgical tool 104.

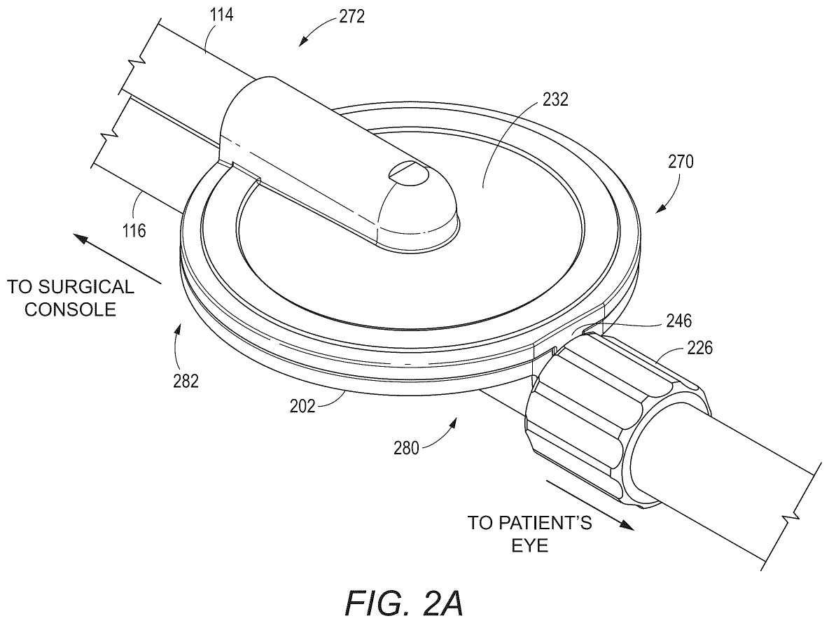

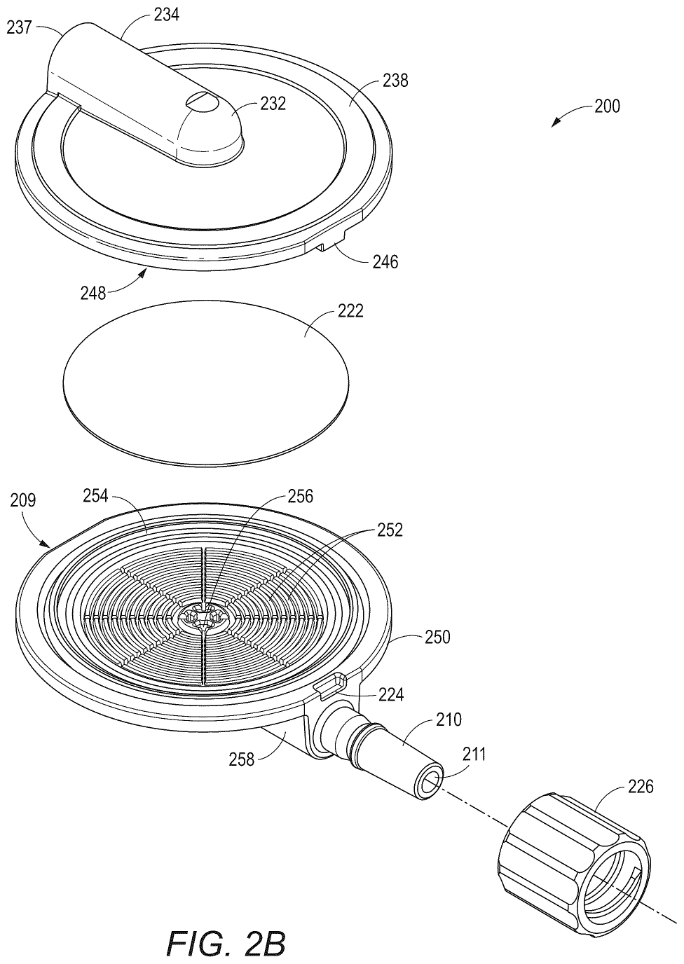

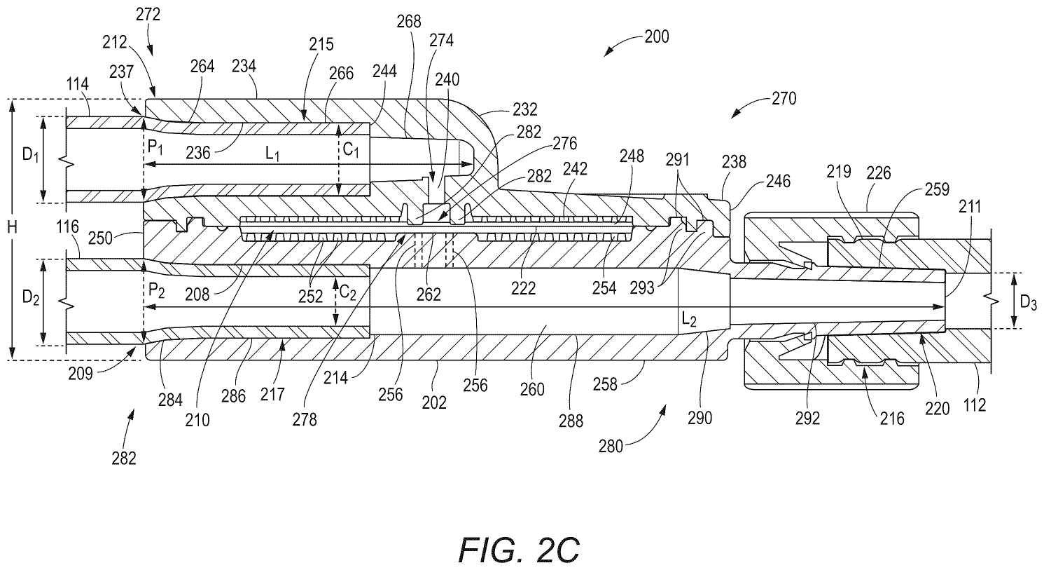

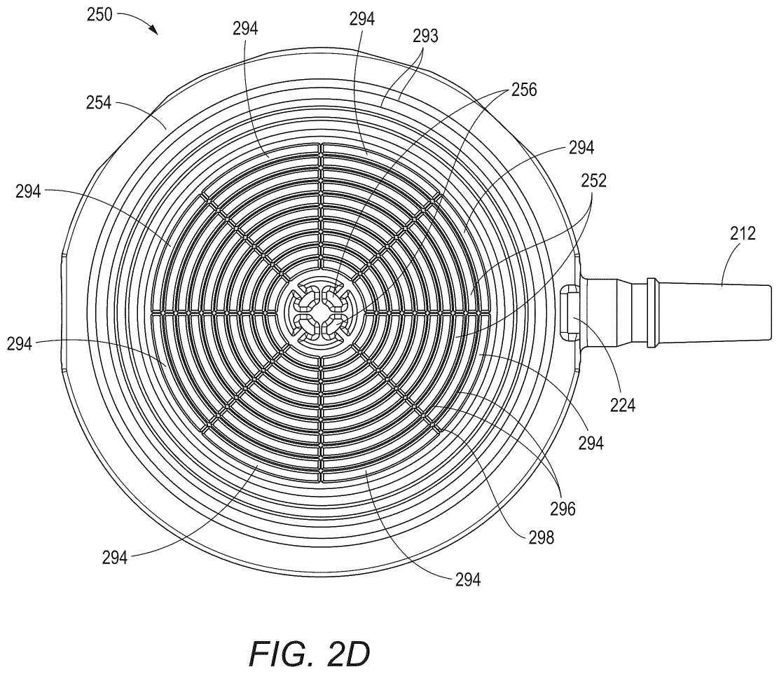

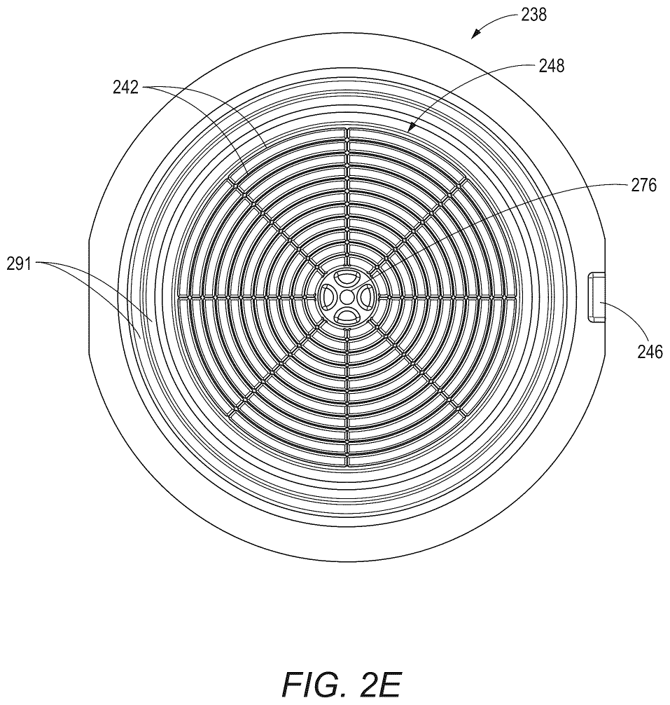

FIGS. 2A-2D illustrate a valve assembly 200 for flow control of infusion fluids during surgical procedures. Valve assembly 200 is an example of the three-way automatic valve assembly 118, which may be utilized in combination with the fluid infusion system 110, surgical tool 104, and the surgical console 100 described above. The valve assembly 200 generally includes a hydrophobic filter (shown as hydrophobic filter 222 in FIGS. 2B-2C) disposed between a first portion (e.g., an upper body) of the valve assembly 200, which is configured to fluidly couple with a gas supply line, and a second portion (e.g., a lower body) of the valve assembly 200, which is configured to fluidly couple with a fluid supply line. Partitioning (e.g., separation) of a volume between the first portion and the second portion by the hydrophobic filter enables active bi-directional flow of gases, such as air, between the gas supply line and an infusion line while passively preventing liquids from traveling into the gas supply line. Thus, gases may be vented or purged from the fluid infusion system 110 during fluid infusion to enable improved control of intraocular pressure during surgical procedures.

FIG. 2A illustrates a perspective view of the valve assembly 200; FIG. 2B illustrates a perspective exploded view of the valve assembly 200; FIG. 2C illustrates a cross-sectional view of the valve assembly 200; FIG. 2D illustrates a perspective top view of the second portion (e.g., lower body) of the valve assembly 200; and FIG. 2E illustrates a perspective bottom view of the first portion (e.g., upper body) of the valve assembly 200. FIGS. 2A-2E are described together herein for clarity.

As noted above, the valve assembly 200 generally includes an upper body 232 configured to interface (e.g., couple) with a lower body 202. In certain embodiments, the upper body 232 and/or lower body 202 are formed of any suitable plastic or thermoplastic materials, such as acrylonitrile butadiene styrene (ABS), polycarbonate (PC), nylon, and acrylic, which may be transparent or opaque in color. In certain embodiments, the upper body 232 and lower body 202 are formed of the same material, while in other embodiments, upper body 232 and lower body 202 are formed of different materials.

In certain embodiments, a distal end 270 of the upper body 232 may include a key 246 that mates with a cutout 224 formed at a distal end 280 of the lower body 202 to ensure that the upper body 232 and lower body 202 are correctly (e.g., rotationally and laterally) aligned when the valve assembly 200 is assembled. Although a single key 246 and cutout 224 are shown, additional keys and/or cutouts may also be formed in the upper body 232 and/or lower body 202 to facilitate alignment between upper body 232 and lower body 202, such as along perimeters thereof. Further, the disposition of the key 246 and cutout 224 may be switched between the upper body 232 and the lower body 202, such that the upper body 232 includes the cutout 224 and the lower body includes the key 246.

Turning to FIG. 2C, in certain embodiments, the upper body 232 and the lower body 202 are configured such that, when assembled, the valve assembly 200 has a height H that is not substantially greater than the combined outer diameters (or widths) D₁ and D₂ of the fluid supply line 116 and the gas supply line 114, respectively. For example, the height H of the valve assembly 200 may be less than 150%, 140%, 130%, 120%, or 110% or less, than a combined outer diameter or width (e.g., D₁+D₂) of the fluid supply line 116 and the gas supply line 114.

The upper body 232 includes a cover 238 from which an arm 234 extends in a proximal direction (e.g., toward a surgical console or gas source, and toward proximal end 272 of the upper body 232) for coupling with gas supply line 114. The gas supply line 114 couples with a port 237 at a proximal end 212 of the arm 234, which provides fluid connection with a conduit 236 extending through a length L₁ of the arm 234. In certain embodiments, the conduit 236 comprises a first tapered portion 264 at a proximal end of the conduit 236 and having a non-uniform diameter (or width), a second tapered portion 268 at a distal end of the conduit 236 and having a non-uniform diameter (or width), and a uniform portion 266 disposed between the first tapered portion 264 and the second tapered portion 268 and having a uniform diameter (or width).

In certain embodiments, a diameter (or width) P₁ of the port 237 at the proximal end 212 of the arm 234 is slightly smaller, substantially the same, or slightly larger than the outer diameter D₁ of the gas supply line 114 to allow a distal end 215 of the gas supply line 114 to be securely fit within the port 237. In certain embodiments, the diameter P₁ of the port 237 is greater than a diameter C₁ of the uniform portion 266 of the conduit 236, such that the first tapered portion 264 forms a sloped, decreasing transition in diameter between the port 237 and the uniform portion 266. Accordingly, the diameter of the first tapered portion 264 decreases in a distal direction from the proximal end 212 of the arm 234. Similarly, in certain embodiments, the diameter of the second tapered portion 268 decreases in a distal direction from the proximal end 212 of the arm 234.

In certain embodiments, the arm 234 includes a step 244 formed between the uniform portion 266 and the second tapered portion 268 of the conduit 236. As a result of the step 244, there is a sudden and abrupt decrease in diameter between a distal end of the uniform portion 266 and a proximal end of the second tapered portion 268. In certain embodiments, the step 244 provides a manual stop for the gas supply line 114, which enables the user (e.g., a surgeon) to consistently insert the gas supply line 114 into the conduit 236 along a predetermined distance for each procedure.

A proximal end 274 of a channel 240 fluidly couples with the second tapered portion 268 of the conduit 236. While the channel 240 extends from the second tapered portion 268, the channel 240 does not, in certain embodiments, extend from a most distal end of the second tapered portion 268; rather, the channel 240 extends from a position between a most proximal end and the most distal end of the second tapered portion 268. As shown, the most distal end of the second tapered portion 268 may have a slightly rounded, or dome-shaped morphology distally beyond the channel 240.

The channel 240 extends from the second tapered portion 268 along an axis that is non-parallel to an axis of the conduit 236. In certain embodiments, the channel 240 is disposed along an axis that is normal to, or disposed at an angle of 90° relative to, the axis of the conduit 236. In certain embodiments, the axis of the channel 240 is disposed at an angle greater than or less than 90° relative to the axis of the conduit 236.

In certain embodiments, the channel 240 has a diameter (or width) less than any diameter or width of the port 237 and the conduit 236. In certain embodiments, the channel 240 has a uniform, or consistent, diameter from the proximal end 274 of the channel 240 nearest the second tapered portion 268 to a distal end 276 of the channel 240 furthest from the second tapered portion 268. In certain embodiments, the channel 240 has a non-uniform diameter from the proximal end 274 to the distal end 276. For example, the channel 240 may comprise one or more steps or sloped transitions in diameter between the proximal end 274 and the distal end 276. As shown in FIG. 2C, in certain embodiments, a diameter of the channel 240 at the distal end 276 is greater than a diameter of the channel 240 at the proximal end 274.

As shown, the upper body 232 of the valve assembly 200 further includes a cover 238, which generally has a disc-like shape and further includes one or more ridges (e.g., ribs or grooves) 242 extending from a lower surface thereof that form one or more channels within a cavity 248. In certain embodiments, the ridges 242 are annular or semi-annular ridges that circumscribe the distal end 276 of the channel 240. The ridges 242 provide added mechanical support for the filter 222 when the valve assembly 200 is in an assembled state.

In certain embodiments, the decreased diameter of the channel 240 relative to the conduit 236 may be configured to increase the localized velocity of gas supplied from the gas supply line 114 as the gas enters the cavity 248 and contacts the filter 222 positioned at the bottom of the cover 238. The additional velocity created by the decreased diameter of the channel 240 may provides enough force on the filter 222 to create a “diaphragm” effect on the filter 222 during use, as discussed elsewhere herein.

The lower body 202 of the valve assembly 200 includes a base 250 coupled to a flow-through member 258. The flow-through member 258 comprises a port 209 at a proximal end 218 of the flow-through member 258 for coupling with the fluid supply line 116, and which further provides fluid connection with a conduit 208 extending through a length L₂ of the flow-through member 258 between the proximal end 218 and a distal end 220 of the flow-through member 258.

In certain embodiments, the conduit 208 comprises a first tapered portion 284 at a proximal end of the conduit 208 and having a non-uniform diameter (or width), a second tapered portion 290 at a distal end of the conduit 208 and having a non-uniform diameter (or width), a third tapered portion 292 at a distal end of the conduit 208 and having a non-uniform diameter (or width), a first uniform portion 286 disposed distal to the first tapered portion 284 and having a uniform diameter (or width), and a second uniform portion 288 disposed between the first uniform portion 286 and the second tapered portion 290 and having a uniform diameter (or width).

In certain embodiments, a diameter P₂ of the port 209 is slightly smaller, substantially the same, or slightly larger than the outer diameter D₂ of the fluid supply line 116 to allow a distal end 217 of the fluid supply line 116 to be securely fit within the port 209. In certain embodiments, the diameter P₂ of the port 209 is greater than a diameter C₂ of the first uniform portion 286 of the conduit 208, such that the first tapered portion 284 forms a sloped, decreasing transition in diameter between the port 209 and the first uniform portion 286. Accordingly, the diameter of the first tapered portion 284 decreases in a distal direction from the proximal end 218 of the flow-through member 258. Similarly, in certain embodiments, the diameters of the second tapered portion 290 and the third tapered portion 292 decrease in a distal direction from the proximal end 218 of the flow-through member 258.

In certain embodiments, the proximal end 218 of the flow-through member 258 may include a step 214 formed between the first uniform portion 286 and the second uniform portion 288. As a result of the step 214, there is a sudden and abrupt decrease in diameter between a distal end of the first uniform portion 186 and a proximal end of the second uniform portion 288. In certain embodiments, the step 214 provides a manual stop for the fluid supply line 116, which enables the user (e.g., a surgeon) to consistently insert the fluid supply line 116 into the conduit 208 along a predetermined distance for each procedure. Further, the step 214, in addition to the second tapered portion 290 and the third tapered portion 292, may be configured to improve fluidics through the lower body 202 and/or to ensure the liquid entering the eye is free of unwanted air bubbles.

The distal end 220 of the flow-through member 258 generally sized to have an outer diameter slightly smaller than an inner diameter of the proximal end of the infusion line 112, and that includes the third tapered portion 292 of the conduit 208 formed therethrough. Accordingly, the infusion line 112 is configured to securely fit around the mating feature 259. In certain embodiments, the distal end of the flow-through member 258 may further include a locking mechanism disposed around the mating feature 259, such as a Luer lock 226, that is configured to provide additional mechanical holding force to secure the infusion line 112 to the valve assembly 200 and create a leak free seal between the mating feature 259 and the infusion line 112. For example, the Luer lock 226 may comprise a threaded interior surface 216 through which the proximal end 219 of the infusion line 112 may be secured within.

The base 250 of the lower body 202 is configured to interface and engage with the cover 238 of the upper body 232 and retain the filter 222 therebetween. In certain embodiments, the base 250 couples to the upper body 232 at a lower surface of the cover 238 such that the cavity 248 of the cover 238 faces cavity 254 of the base 250. When valve assembly 200 is fully assembled, the filter 222 partitions, or separates, the cavities 248 and 254.

In certain embodiments, the base 250 has one or more mating features 293 formed on an upper surface thereof that are configured to mate, or engage with, corresponding mating features 291 formed on a lower surface of the cover 238 when the valve assembly 200 is assembled. Such mating features 293 and 291, in addition to the key 246 and cutout 224, may facilitate lateral and/or rotational alignment of the lower body 202 with the upper body 232. In certain embodiments, the mating features 293 and 291 are disposed radially outward of the cavities 254 and 248, respectively, and radially inward of an outer circumference of the base 250 and cover 238, respectively. In certain embodiments, as best seen in FIGS. 2D-2E, the mating features 293 and 291 are annular, and follow along an entire outer circumference of the base 250 and cover 238, respectively. In certain embodiments, each of the mating features 293 and 291 are semi-annular and follow along only a portion of the outer circumference (e.g., along 25% of the circumference, or along 50% of the circumference) of the base 250 and cover 238, respectively. In such embodiments, the mating features 293 and 291 may each comprise a plurality of features arranged in a circumferential manner and separated by one or more gaps. Generally, the mating features 293 may comprise grooves, protrusions, and/or other similar features, while mating features 291 may comprise corresponding grooves, protrusions, and/or other similar features configured to mate with mating features 293.

Similar to the cover 238, the base 250 includes one or more ridges 252 extending from an upper surface thereof into a cavity 254. The ridges 252 are configured to provide mechanical support to the filter 222 when the valve assembly 200 is in an assembled state. The ridges 252 may be densely populated within the base 250, such that there is very little volume within the base 250 not occupied by ridges 252. The dense positioning of ridges 252 within the base 250 may limit the volume available within the cavity 254 for fluid to enter during fluid-air exchange operations.

Generally, the ridges 252 may have any suitable shape and/or arrangement within the cavity 254. In certain embodiments, as shown in FIG. 2D, the ridges 252 are semi-annular and are disposed in concentric rings 294 that increase in size (e.g., diameter/circumference) with increased distance of the concentric ring 294 from a center 262 of the base 250. The concentric rings (e.g., see concentric ring 294 in FIG. 2D) of ridges 252 form corresponding concentric rings of channels 296 between adjacent rings of ridges 252.

Further, in embodiments with semi-annular ridges 252 arranged in concentric rings 294, the semi-annular ridges 252 in each concentric ring 294 may increase in length with increased distance of the concentric ring 294 from the center 262. In certain embodiments, each concentric ring 294 comprises breaks or gaps 298 between the ridges 252 thereof, and in certain embodiments, the gaps 298 of some or all of the concentric rings 294 may be circumferentially aligned. As shown in FIG. 2D, alignment of the gaps 298 of all the concentric rings 294 in base 250 may form straight channels extending radially outward from the center 262.

Turning to FIG. 2E, the cavity 248 of the upper body 232 may also include semi-annular ridges 242 as discussed herein with reference to FIG. 2D. The semi-annular ridges 242 may be arranged in concentric rings creating corresponding annular channels between each ring of ridges 242, and gaps between the ridges 242 may form straight channels through the concentric rings that extend outward from a center of the cavity 248. Though a specific arrangement of ridges and channels is described and shown herein, alternate arrangements of ridges and channels the cavity 248 and/or cavity 254 are also contemplated.

Returning to FIG. 2C, in an assembled state, the valve assembly 200 may comprise a clearance space 210 between the ridges 252 of the base 250 and the ridges 242 of the cover 238. In certain embodiments, the clearance space 210 may be 0.025 inches to 0.05 inches in height when the valve assembly 200 is in an assembled state. In certain embodiments, the height of clearance space 210 is optimized to allow a controlled fluid volume to enter the base 250 and occupy the clearance space 210. The clearance space 210 may be further configured to allow for the movement of the filter 222 between the ridges 252 of the basin and the ridges 242 of the cover 238 and vice versa during fluid-air exchange operations as further described below.

In certain embodiments, the ridges 252 circumscribe proximal ends of a plurality of through channels (e.g., ports) 256 that fluidly couple the cavity 254 with the conduit 208 of the flow-through member 258. Accordingly, the port 237 at the proximal end 272 of the upper body 232 is fluidically coupled to the port 211 at the distal end 280 of the lower body 202 via the combination of the conduit 236, the channel 240, the cavity 248, the flow through channels 256, the cavity 254, and the conduit 208.

The through channels 256 may include two or more channels disposed through a center 262 of the base 250 and fluidly coupling the cavity 254 with the conduit 208. A proximal end of each through channel 256 is disposed adjacent to the base 250, while a distal end of each through channel 256 is disposed adjacent to the conduit 208. In certain embodiments, the through channels 256 may include two, three, four, five, or more through channels 256 arranged in a circular arrangement through the base 250 (four through channels 256 are shown in FIG. 2D). In certain embodiments, the through channels 256 may each have a curved and/or oblong shape, as pictured in FIG. 2D. In certain other embodiments, the through channels 256 may be a circular, oval, or polygonal shape. In certain embodiments, each through channel 256 is uniform in lateral dimensions and/or shape through the base 250 (e.g., the proximal and distal ends of each through channel 256 may have the same dimensions). In certain embodiments, each through channel 256 is non-uniform in lateral dimensions and/or shape through the base 250 (e.g., the proximal and distal ends of each through channel 256 may have different dimensions). In certain embodiments, one through channel 256 of the plurality of through channels 256 may be different in dimensions and/or shape from another through channel 256 of the plurality of through channels 256.

In certain embodiments, the center 262 of the base 250 includes a raised support member 278 disposed around portions of the proximal ends of the through channels 256 to support a central region of the filter 222. The support member 278 may also be configured to control the evacuation of the infusion fluid from the cavity 254 (e.g., by distributing fluid flow through each of the two or more cavities) during the fluid-air exchange as described herein. In certain embodiments, the support member 278 comprises a plurality of walls arranged in a crutch cross, anchor cross, or cercelée cross-like shape around the through channels 256 and radially inward of the ridges 252.

In certain embodiments, the cover 238 has one or more raised support members 282 surrounding the distal end 276 of the channel 240. The raised support members 282 may include one or more raised features having any suitable shape and arranged in any suitable arrangement around the distal end 276 of the channel 240. The support members 282 may be configured to support the center of the filter during a fluid-air exchange operation, similar to support member 278.

The filter 222, disposed between the upper body 232 and lower body 202 may include any suitable type of membrane filter having a hydrophobic membrane permeable to gas. The hydrophobic membrane may also be capable of capturing individual viruses and bacteria, thus acting as a sterile barrier (e.g., filter) to prevent viruses and bacteria from entering the eye from the low pressure gas force.

In some examples, the filter 222 includes a membrane formed of polytetrafluoroethylene (PTFE), expanded PTFE (ePTFE), polycarbonate track etch (PCTE), polyesters (e.g., polyethylene terephthalate (PET)), nylon, cellulose (e.g., surfactant free cellulose acetate (SCFA), cellulose nitrate (CN), cellulose acetate (CA), polyethersulfone (PES), glass fibers, or acrylic copolymers. The membrane may further be unsupported or supported by a backing formed of materials including but not limited to polyester, polyethylene, polypropylene, or nylon. For example, in certain embodiments, the filter 222 includes an ePTFE membrane having a polyester backing. The membrane has a pore size ranging between about 0.1 μm (micrometer) to about 10.0 μm, such as between about 0.2 μm to about 5 μm, such as between about 0.5 μm to about 3.0 μm, such as between about 0.8 μm to about 1.2 μm. Furthermore, the membrane may have a thickness ranging between about 150 μm to about 300 μm, such as between about 200 μm to about 250 μm.

During operation of the valve assembly, infusion liquid from the liquid source may flow through the liquid supply line 116, into the flow-through member 258 of the lower body 202, and through the infusion line 112 toward the patient's eye and vice versa. Alternatively, infusion gases from the gas source may flow through the gas supply line 114, into the upper body 232, past the filter 222 into the lower body 202, and through the infusion line 112 toward the patient's eye and vice-versa. The disposition of the hydrophobic filter 222 between the upper body 232 and the lower body 202 passively prevents the flow of liquids into the upper body 232 and the gas supply line 114, while allowing gases to pass therethrough. Thus, the valve assembly 200 facilitates venting, purging, and/or back-flow of gases during fluid infusion procedures while preventing the escape of liquid into the gas supply line 114.

During the fluid-air exchange, the infusion liquid may flow through the liquid supply line 116 and the flow-through member 258 of the lower body 202, while also flowing through the through channels 256 and into the cavity 254. The infusion liquid may occupy the volume between the ridges 252 and push the filter 222 upwards towards the cover 238, until the filter is pressed against the ridges 242 of the cover 238. When infusion gases from the gas supply line 114 reach the cavity 248 and contact the filter 222, the infusion gases cause the filter 222 to “diaphragm” down (e.g., push downwards) towards the ridges 252 of the base 250, thereby pushing infusion liquid through the channels in the ridges 252 to the through channels 256 and into the flow through member 258.

Please note that although a single filter 222 is depicted in FIGS. 2A-2C, it is further contemplated that the valve assembly 200 may include two or more filters arranged in a linear or stacked configuration. The two or more filters may be formed of different materials and/or have different pore sizes relative to each other. For example, in certain embodiments, a second filter having a finer pore size may be disposed upstream of the filter 222 to provide additional filtration of gases flowed through the gas supply line 114, while the filter 222 provides a hydrophobic barrier and prevent liquids from flowing therein.

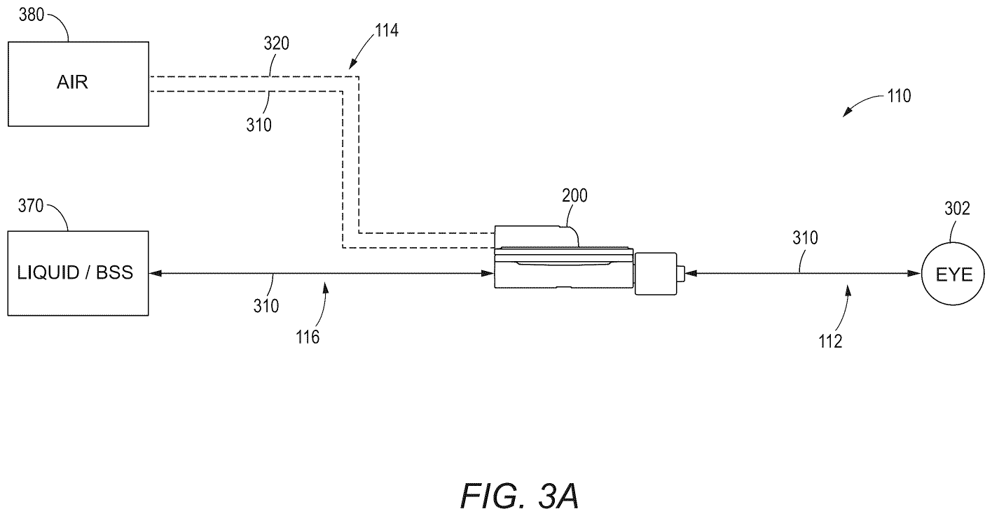

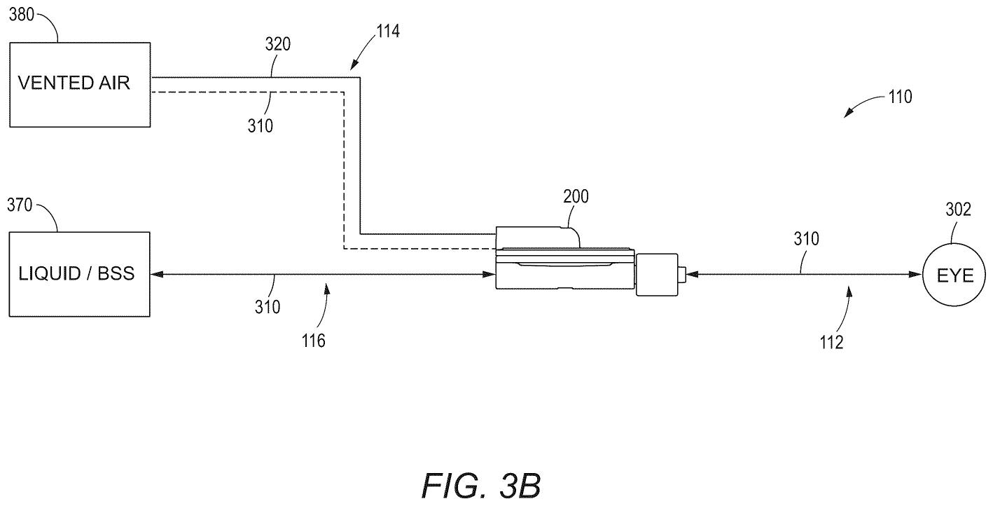

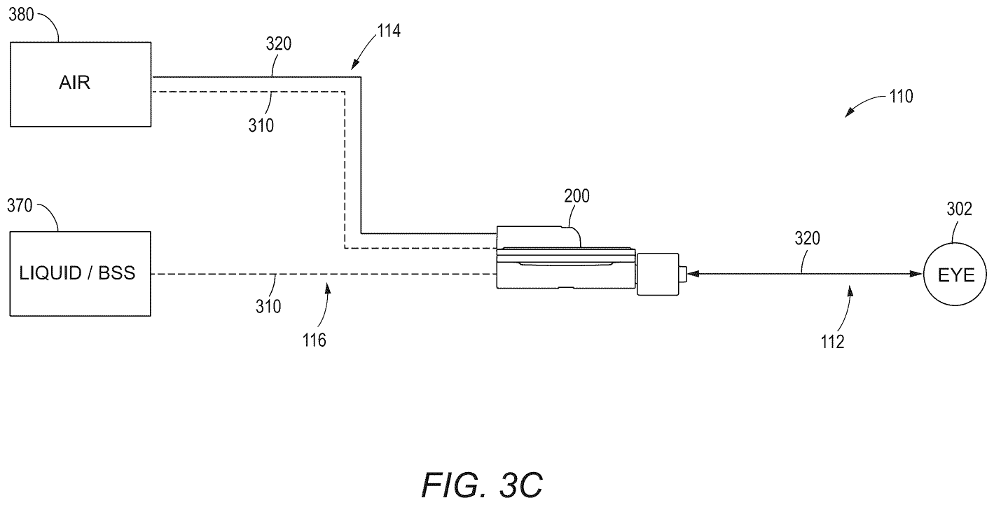

FIGS. 3A-3C schematically illustrate operational modes of the valve assembly 200 during fluid infusion procedures. In particular, FIGS. 3A-3C illustrate the flow of liquid solutions (e.g., BSS), represented by lines 310, and the flow of gases (e.g., air), represented by lines 320, through the fluid infusion system 110 having the valve assembly 200, as described above. Further, please note that unbroken lines (e.g., continuous lines) represent open or active flow, while broken lines (e.g., dashed lines) represent closed or no flow.

FIG. 3A depicts the fluid infusion system 110 during a first operation of liquid infusion, which may be selected and/or controlled by a user (e.g., a surgeon) via a surgical console, such as surgical console 100. As shown, infusion liquid 310 is controllably flowed between the liquid source 370 and eye 302 via liquid supply line 116, valve assembly 200, and infusion line 112, while air or gas flow through gas supply line 114 is stopped or shut off. To control a pressure within the fluid infusion system 110 and thus, the eye 302, the user may adjust the direction and flow rate of the liquid 310 to or from the liquid source 370 via the surgical console 100. The valve assembly 200 enables liquid 310 to flow between the liquid supply line 116 and the infusion line 112, while also preventing the liquid 310 from flowing into the gas supply line 114 and towards the gas source 380 due to the presence of the hydrophobic filter 222. Accordingly, the valve assembly 200 provides a passive means of preventing leakage of liquid 310 into gas supply line 114, which contrasts with conventional flow control valves that may allow the escape of at least some liquid 310 into the gas supply line 114 during use thereof.

FIG. 3B depicts the fluid infusion system 110 during a second operation of liquid infusion in which the pressure of air 320 within the gas supply line 114 is actively modulated while infusion liquid 310 is flowed between the liquid source 370 and eye 302. As described above, the pressure within the fluid infusion system 110 and the eye 302 is controlled by adjusting the direction and flow rate of the liquid 310 to or from the liquid source 370 via the surgical console 100. When left unchecked, pressure within the gas supply line 114 may inadvertently build up during infusion and cause air 320 to leak into the liquid 310 being injected into the eye 302, thereby negatively affecting the intraocular pressure thereof. Therefore, in certain embodiments, it may be desired to apply a vacuum pressure (e.g., negative pressure) to the gas supply line 114 to vent the gas supply line 114 and prevent the undesired escape of air 320 into the liquid 310 as bubbles. In certain embodiments, active venting of the gas supply line 114 may also be desired to purge the infusion liquid 310 of gases already trapped therein as the liquid 310 passes into the infusion line 112.

Since conventional flow control valves cannot prevent the leakage of liquid 310 into the gas supply line 114, venting of the gas supply line 114 with a conventional valve is extraordinarily difficult. In comparison, as a result of the hydrophobic filter 222, the valve assembly 200 facilitates active venting of the gas supply line 114 during infusion of liquid 310 into the eye 302, thus reducing or eliminating the possibility of unwanted gases being flowed into eye 302 and disrupting the intraocular pressure therein.

FIG. 3B is further representative of the fluid infusion system 110 during an infusion fluid back-flow operation. Back-flow of infusion fluids may be necessitated when the eye 302 is injected, via a separate cannula or injection device, with a retinal tamponade (or other fluid treatment) such as intraocular air/gas, silicone oil, or perfluoron. As a result, infusion fluids previously flowed through the infusion line 112 may need to be back-flowed. Because conventional flow control valves cannot backflow or purge gases into the gas supply line 114 without leakage of infusion liquid, only a limited volume of infusion fluids can be back-flowed without risking the chance of liquid leakage into the gas supply line 114 or gas leakage into the liquid supply line 116. In contrast, the hydrophobic filter 222 of the valve assembly 200 in FIG. 3B enables backflow of gases into the gas supply line 114 without leakage of infusion liquids, thus allowing a greater volume of the infusion fluids to be back-flowed into their respective supply lines and further enabling a greater volume of treatment fluids to be injected into the eye 302.

FIG. 3C depicts the fluid infusion system 110 during a third operation of liquid infusion. The operational mode depicted in FIG. 3C may be performed, for example, during a fluid-air exchange to help push out subretinal fluid from the intraocular space of the eye 302. As shown, air 320 is flowed from the gas source 380 to the eye 302, while liquid flow through the liquid supply line 116 is shut off to prevent escape of liquid 310 into the infused air 320. Accordingly, the pressure within the fluid infusion system 110 and the eye 302 is controlled by adjusting the direction and flow rate of the air 320 to or from the gas source 380 via the surgical console 100.

In summary, embodiments of the present disclosure include structures and mechanisms for improved intraocular pressure maintenance during ophthalmic procedures, and in particular, improved fluid control valves for intraocular fluid infusion. The valve assemblies described above include embodiments wherein a hydrophobic filter is disposed between a gas supply line and a liquid supply line and/or infusion line. The utilization of the hydrophobic filter enables bi-directional flow of gases between a gas supply line and the patient's eye, while also passively preventing the leakage of liquids into the gas supply line. Accordingly, the aforementioned valve assemblies are particularly beneficial during fluid infusion of the intraocular space, as gas may be vented from infusion liquids during infusion or black-flowed from the eye during injection of other treatments, thus allowing better control of the intraocular pressure within the eye.

The many features and advantages of disclosure are apparent from detailed specification, and, thus, it is intended by appended claims to cover all such features and advantages of disclosure which fall within scope of disclosure. Further, since numerous modifications and variations will readily occur to those skilled in art, it is not desired to limit disclosure to exact construction and operation illustrated and described, and, accordingly, all suitable modifications and equivalents may be resorted to that fall within scope of disclosure.

What is claimed is:

1. A valve assembly for fluid infusion during ophthalmic procedures, comprising:
a first portion, comprising:
a first conduit having a first port; and
a first cavity having a first end and a second end opposite the first end, the first end fluidly coupled to the first port via a first conduit, the second end having a diameter less than the diameter of the first conduit;
a second portion, comprising:
a second conduit having a second port and a third port; and
a second cavity in fluid communication with the second port and the third port, the second cavity adjacent to the first cavity, wherein two or more through channels couple the second cavity to the second port and the third port; and
a filter partitioning the first cavity from the second cavity, the filter comprising a hydrophobic membrane partially defining the second cavity.

2. The valve assembly of claim 1, wherein the two or more through channels are circular, oval, or polygonal channels.

3. The valve assembly of claim 2, wherein the two or more through channels are configured in a circumferential pattern around a center of a bottom surface of the second cavity.

4. The valve assembly of claim 1, wherein the hydrophobic membrane is configured to prevent a flow of liquid from the second cavity into the first cavity while allowing bi-directional flow of gas therebetween.

5. The valve assembly of claim 1, wherein the filter is configured to translate upwards in response to a fluid entering the second cavity from the second conduit, and subsequently translate downwards against the fluid in response to a gas entering the first cavity from the first conduit.

6. The valve assembly of claim 1, wherein the first cavity further comprises one or more semi-annular or annular ridges disposed therein, the one or more ridges defining one or more channels.

7. The valve assembly of claim 6, wherein the second cavity further comprises one or more semi-annular or annular ridges disposed therein, the one or more ridges defining one or more channels.

8. The valve assembly of claim 7, wherein the one or more ridges of the first cavity and the second cavity comprise at least ten or more semi-annular or annular ridges defining one or more channels.

9. A fluid infusion system for ophthalmic procedures, comprising:
a surgical console, comprising:
a first fluid line coupled to a gas fluid source; and
a second fluid line coupled to a liquid fluid source; and
a valve assembly fluidly coupled to the first fluid line and the second fluid line, the valve assembly comprising:
a first conduit having a first port directly coupled to the first fluid line;
a second conduit having a distal end and a proximal end, wherein the proximal end has a second port directly coupled to the second fluid line and the distal end has a third port coupled to a third fluid line, wherein:
the first and second conduit are coupled to an intermediary cavity and are in fluid communication with each other through two or more channels; and
the surgical console controls flow rates of fluids through the first fluid line, the second fluid line, the third fluid line, and the valve assembly; and
a filter disposed within the intermediary cavity, the filter partitioning the first conduit from the second conduit.

10. The fluid infusion system of claim 9, wherein the two or more channels are circular, oval, or polygonal channels.

11. The fluid infusion system of claim 10, wherein the two or more channels are configured in a circumferential pattern around a center of a bottom surface of the intermediary cavity.

12. The fluid infusion system of claim 9, wherein the filter comprises a hydrophobic membrane disposed on a side thereof and configured to prevent the flow of liquid from the second conduit into the first conduit while allowing bi-directional flow of gases therebetween.

13. The fluid infusion system of claim 9, wherein the filter is configured to translate upwards in response to a fluid entering the intermediary cavity from the second fluid line via the second conduit, and subsequently translate downwards against the fluid in response to a gas entering the intermediary cavity from the first fluid line via the first conduit.

14. The fluid infusion system of claim 9, wherein the intermediary cavity further comprises one or more semi-annular or annular ridges disposed on a top surface and a bottom surface adjacent to one another, the one or more ridges defining one or more channels.

15. The fluid infusion system of claim 14, wherein the one or more ridges comprise at least ten or more semi-annular or annular ridges defining one or more channels.
